(19) United States
(12) Patent Application Publication
(10) Pub. No.: US 2018/0221583 A1
(43) Pub. Date: Aug. 9, 2018

(54) DEVICE FOR ADMINISTERING AN ACTIVE SUBSTANCE AND METHOD FOR OPERATING SAID DEVICE

(71) Applicant: TECPHARMA LICENSING AG, Burgdorf (CH)

(72) Inventors: Susanne Schenker, Langenthal (CH); Ursina Streit, Schönbühl (CH)

(21) Appl. No.: 15/944,054

(22) Filed: Apr. 3, 2018

Related U.S. Application Data
(63) Continuation of application No. PCT/CH2016/000129, filed on Oct. 4, 2016.

(30) Foreign Application Priority Data
Oct. 19, 2015 (CH) 01508/15

(51) Int. Cl. A61M 5/24; A61M 5/31; A61M 5/315

(52) U.S. Cl. CPC A61M 5/2448; A61M 5/3137; A61M 5/31511; A61M 5/19; A61M 2005/31508; A61M 5/3155; A61M 2005/2451

(57) ABSTRACT

A device for administering an active substance comprises a multi-chamber carpule having a bypass in one wall and at least one first chamber with a solid active substance and a second chamber with a solvent for the active substance, the second chamber being delimited by a first plug arranged distally in the multi-chamber carpule and a second plug arranged proximally therein; a mixing element is rotatably mounted about a rotational axis in relation to the housing and can be rotated about the rotational axis in a rotational direction in order to mix the active substance and the solvent; and a first detent device is provided on the device such that, after the mixing operation, an axial displacement of the distal end of the second plug in relation to the wall of the multi-chamber carpule is blocked in the proximal direction by means of the distal end of the bypass.

### CROSS-REFERENCE TO RELATED APPLICATIONS

This application is a continuation of International Application No. PCT/CH2016/00129 filed Oct. 4, 2016, which claims priority to Swiss Application No. 01508/15 filed Oct. 19, 2015, the entire contents of all of which are incorporated by reference herein in their entirety.

### BACKGROUND

The invention relates to a device for administering an active substance and to a method for operating said device. The device comprises a multi-chamber carpule with a solid active substance and a solvent, and a mixing device for mixing the active substance and the solvent, wherein the mixing device comprises a mixing element and a bendable piston rod, which are coupled to one another.

Such a device is used to make it possible for a solid substance that is unstable in a fluid to be mixed with a solvent only shortly before the administration and that can consequently be stored. Such devices are known from the prior art, for example, from WO2008/122132 A1 and WO2008/122360 A2.

An example of a solid active substance that is unstable in a fluid and mixed with a solvent only shortly before the administration is a recombinantly produced coagulation factor in the form of a lyophilizate, which is used for treating hemophilia.

Hemophilia is a hereditary disease that leads to impaired blood coagulation. The blood of affected patients does not coagulate or it coagulates after a delay. Therefore, spontaneous bleeding can also occur, without any visible wounds.

The treatment of patients with hemophilia generally consists in substituting the respective missing or defective factor. By the regular administration of coagulation factors it is ensured that their concentration does not drop below a critical value. In most patients, the occurrence of dangerous spontaneous bleeding can thus be largely prevented. For the therapy, the patients regularly inject a certain quantity of coagulation factors into a vein, for example, several times per week.

In the context of a substitution therapy, patients with hemophilia A receive injections of the lacking coagulation factor VIII approximately two to three times per week. Patients with hemophilia B need coagulation factor IX. After appropriate training, the patients themselves can administer the appropriate coagulation factors by means of an administration apparatus.

In the case of administration of substituted coagulation factors in the form of a lyophilizate, in particular, the challenge is that after the dissolution of the lyophilized coagulation factor in a solvent and after sticking a cannula of an infusion set, which is attached to an administration device, into a vein of the patient, it must be possible to aspirate, that is to say, suction the venous blood through the cannula of the infusion set. Through a transparent tube of the infusion set, the patient can see whether the cannula is located in a vein, since, due to aspiration, the color of the suctioned venous blood shines through the transparent tube.

Such devices are known from the prior art.

WO2013/149032 A1 describes a device for mixing lyophilized coagulation factors with a solvent and for administering dissolved coagulation factors into a vein of a patient with hemophilia. During the mixing operation, a mixing device is moved into a distal direction of the administration device.

WO2013/048877 A1 describes a device for administering an active substance, wherein the device comprises a bendable piston rod for dispensing the active substance.

EP721358 B1 describes a device for administering an active substance, wherein the bendable piston rod is driven with a spindle drive.

The disadvantage of the above-mentioned devices is that after the mixing operation of a solid active substance with a solvent, no reliable and simple handling of the aspiration and of the administration of the dissolved active substance can be ensured.

### SUMMARY OF THE INVENTION

Therefore, an aim of the present invention is to provide a device for administering an active substance, which, after the mixing operation of a solid substance with a solution fluid, ensures a reliable and simple handling of the aspiration and of the administration of the dissolved active substance.

Another aim of the present invention is to provide a method for operating a device for administering an active substance, by means of which a mixing of a solid active substance with a solvent, an aspiration and an administration of the dissolved active substance can be carried out in a reliable and simple manner.

These aims are achieved by the subject matter of the independent claims. Advantageous example embodiments are defined in the dependent claims.

Below, distal is understood to mean a direction towards the infusion set-side or cannula-side end of the administration device and proximal is understood to mean a direction towards the opposite end.

The invention relates to a device for administering an active substance, which comprises a multi-chamber carpule having a bypass in a wall of the multi-chamber carpule and at least one first chamber with a solid active substance and a second chamber with a solvent for the active substance. The second chamber is delimited by a first plug arranged distally in the multi-chamber carpule and a second plug arranged proximally in the multi-chamber carpule. The administration device moreover comprises a housing, wherein the multi-chamber carpule is accommodated at least partially by the housing. In addition, the administration device comprises a mixing device for mixing the active substance and the solvent, wherein the mixing device comprises a mixing element, a sliding element and a bendable piston rod. The bendable piston rod is used for a short construction of the administration device. The mixing element is rotatably mounted about a rotational axis in relation to the housing and can be rotated about the rotational axis in a rotational direction in order to mix the active substance and the solvent. After the mixing operation, the sliding element is axially movable back and forth in relation to the housing. The sliding element is coupled to the second plug via the piston rod in such a manner that during the axial back and forth movement of the sliding element in relation to the housing, the second plug can be moved back and forth in relation to the wall of the multi-chamber carpule. The second plug can preferably be connected to the piston rod by means of an axially fixed connection, for example, a gluing, clamping or screw connection.

Furthermore, on the administration device, a first detent device can be provided in such a manner that after the completion of the mixing operation, an axial displacement of a distal end of the second plug in relation to the wall of the multi-chamber carpule is blocked in the proximal direction by means of a distal end of the bypass. The first detent device is used to enable an axial relative movement between the second plug and the housing, in particular the wall of the multi-chamber carpule, only up to an aspiration stop position. The first detent device can ensure that after the mixing operation, the first plug and the second plug are always connected in an air-tight manner to one another. On the other hand, it is possible to prevent that, after the mixing operation of the active substance with the solvent, air and/or the dissolved active substance can reach the area of the bypass of the multi-chamber carpule. Furthermore, the first detent device is used for a reliable and simpler administration of the dissolved active substance.

Particularly preferably, the first detent device can form a first detent position between the housing and the mixing element or sliding element or the piston rod. The first detent device can be formed as an abutment device. The first detent device can be implemented, for example, by a snap arm or detent arm on the mixing element or sliding element or on the piston rod, which can abut against an abutment surface or recess or detent groove on the housing, in order to delimit a relative axial movement between the housing and the mixing element or sliding element or the piston rod. The abutment contact between the snap arm or detent arm and the abutment surface or recess or detent groove can be formed as an axial abutment contact. The snap arm or detent arm can be arranged on the periphery of the mixing element or of the sliding element or of the piston rod. The snap arm or detent arm can extend along a circumferential direction or longitudinal direction and can be radially pretensioned, in particular resiliently radially pretensioned. Alternatively, the housing can comprise the snap arm or detent arm, and the mixing element or the sliding element or the piston rod can comprise the abutment surface or recess or detent groove. Alternatively, in each case, a plurality of snap arms, detent arms, abutment surfaces, recesses or detent grooves can be provided.

Particularly preferably, the abutment surface or the recess or detent groove can be formed as a longitudinal guide, which comprises a distal and a proximal abutment, in order to limit an axial back and forth movement of the sliding element in relation to the housing.

Furthermore, a second detent device can be provided on the device in such a manner that during the mixing of the active substance and the solvent, an axial displacement of the second plug in the proximal direction in relation to the housing, in particular the wall of the multi-chamber carpule, is prevented. The second detent device can be used for the reliable mixing of the solid active substance with the solvent, since a mixing movement in the direction opposite the mixing direction is prevented. A mixing operation that has started can thus not be undone. Furthermore, the second detent device can comprise an indicator device, wherein the indication can be produced by means of an acoustically perceivable detent sound. The second detent device can preferably form a second detent position between the housing and the mixing element or the sliding element or the piston rod. For this purpose, for example, a snap arm or a detent arm can be provided on the mixing element or sliding element or on the piston rod, which can abut against an abutment surface or recess or detent groove on the housing, in order to limit a relative axial movement between the housing and the mixing element or the sliding element or the piston rod. The abutment contact between the snap arm or detent arm and the abutment surface or recess or detent groove can be formed as a radial or axial abutment contact, for example, by means of a safety catch or hook. The snap arm or detent arm can be arranged on the periphery of the mixing element or of the sliding element or of the piston rod. The snap arm or detent arm can extend along a circumferential direction or longitudinal direction and, in particular, it can be radially pretensioned resiliently. Alternatively, the housing can comprise the snap arm, and the mixing element or sliding element or the piston rod can comprise the snap arm. Alternatively, the housing can comprise the snap arm or detent arm, and the mixing element or the sliding element or the piston rod can comprise the abutment surface or recess or detent groove. Alternatively, in each case, a plurality of snap arms, detent arms, abutment surfaces, recesses or detent grooves can be provided.

Particularly preferably, the administration device can comprise one or more second detent devices and/or indicator devices that verify and/or display and/or signal different positions, in particular, the relative position between the sliding element and the housing during the mixing operation. Due to the movement of the sliding element in relation to the housing, first a second detent position and/or indicator in the start position and then in the mixing position and in the final priming position, in the ventilation position and finally in the dispensing position can be verified or indicated. Furthermore, the display device or signal can be formed as a visual, tactile or acoustic indicator.

The snap arm or detent arm of the first detent device can be identical to the snap arm or detent arm of the second detent device. However, different snap arms or detent arms can be provided, which perform the corresponding functions. Moreover, the abutment surface or recess or detent groove of the first detent device can be identical to the abutment surface or recess or detent groove of the second detent device. However, different abutment surfaces or recesses or detent grooves, which perform different functions, can also be provided.

Particularly preferably, the bendable piston rod can be of resilient design. The piston rod can be formed as a band or spring. The piston rod can be formed as a single part or it can be formed from several segments arranged one after the other. The bendable design of the piston rod ensures a short construction of the administration device. The material can be a plastic or a metal or a combination of the two. For example, a metal band made of a spring steel or a spiral-shaped spring made of spring steel can be provided. The housing can comprise a guide slot that can guide the bendable piston rod under tension and compression, in particular, a two-walled saddle guide or a cylindrical deflection cavity or a deflection bore that deflects the piston rod under tension and compression by approximately 180°. Alternatively or additionally, the holding device can comprise such a guide slot for guiding the piston rod or it can form such a guide slot together with the housing.

Particularly preferably, the sliding element can be mounted on the housing in an axially displaceable manner in relation to the multi-chamber carpule. The sliding element can be formed in such a manner that the patient can displace the mixing element axially back and forth with a finger. The sliding element can comprise a recess, wherein a finger of the patient can be accommodated at least partially. The sliding element can be adapted to the shape of the housing or have another shape. Furthermore, the sliding element can comprise longitudinal and/or transverse ribs, so that the patient is better able to firmly hold and/or guide the sliding element.

Preferably, the mixing element can have a thread, and the sliding element can have a counter-thread, wherein the thread and the counter-thread are in threaded engagement, in particular in a self-locking threaded engagement. Moreover, the sliding element can be in a longitudinal guide with the housing. The mixing element, the sliding element and the housing can be coupled to one another in such a manner that when the mixing element is rotated towards the housing, the sliding element can be moved towards the housing axially in relation to the housing. The administration device can provide a gear train or transmission of the mixing movement. Furthermore, for aspiration with the administration device, the sliding element and thus the piston rod and the plugs can be moved axially back and forth in particular in relation to the housing and to the multi-chamber carpule.

Preferably, the sliding element can comprise a gear, which can interact with a toothed surface, which is arranged on a surface of the housing. The administration device can consequently comprise a gear drive, wherein a lever action can be operative. The mixing operation and the aspiration of the administration device can be carried out by the gear drive. The sliding element can also comprise several gears.

Furthermore, the sliding element can be accommodated during the mixing of the active substance and the solvent by the housing and be accessible to the patient only after the mixing operation and/or priming operation.

### BRIEF DESCRIPTION OF THE DRAWINGS

FIG. 1 shows a perspective view of a first embodiment of an administration device.

FIG. 2 shows a perspective view of the first embodiment of an administration device, wherein the administration device can be seen in an exploded view.

FIG. 3a shows a longitudinal sectional view of the first embodiment of the administration device in a start position.

FIG. 3b shows a longitudinal sectional view of the first embodiment of the administration device in a mixing position.

FIG. 3c shows a longitudinal sectional view of the first embodiment of the administration device in a priming position.

FIG. 3d shows a longitudinal sectional view of the first embodiment of the administration device in an aspiration stop position.

FIG. 3e shows a longitudinal sectional view of the first embodiment of the administration device in an end position.

FIG. 4 shows a perspective view of a second embodiment of the administration device.

FIG. 5 shows a perspective view of a third embodiment of the administration device.

FIG. 6 shows a perspective view of a fourth embodiment of the administration device.

FIG. 7a shows a perspective view of a fifth embodiment of the administration device.

FIG. 7b shows a longitudinal sectional view of the fifth embodiment of the administration device according to FIG. 7a.

### DETAILED DESCRIPTION

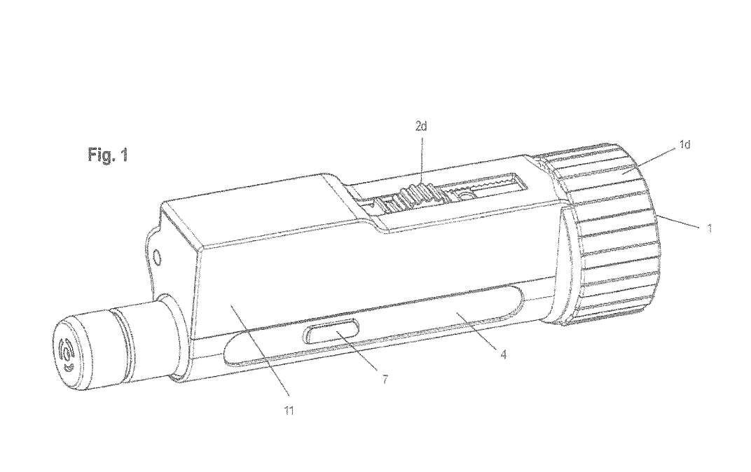

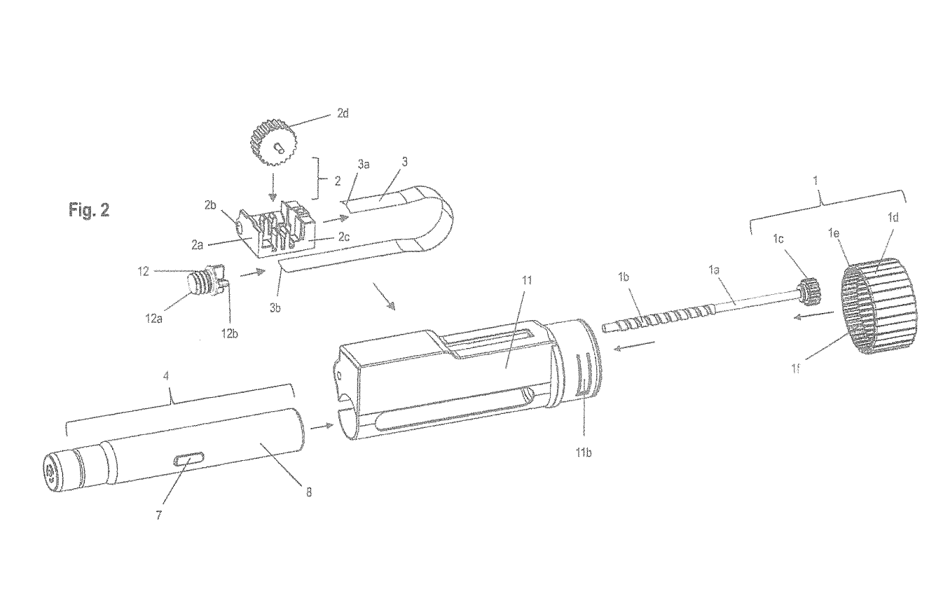

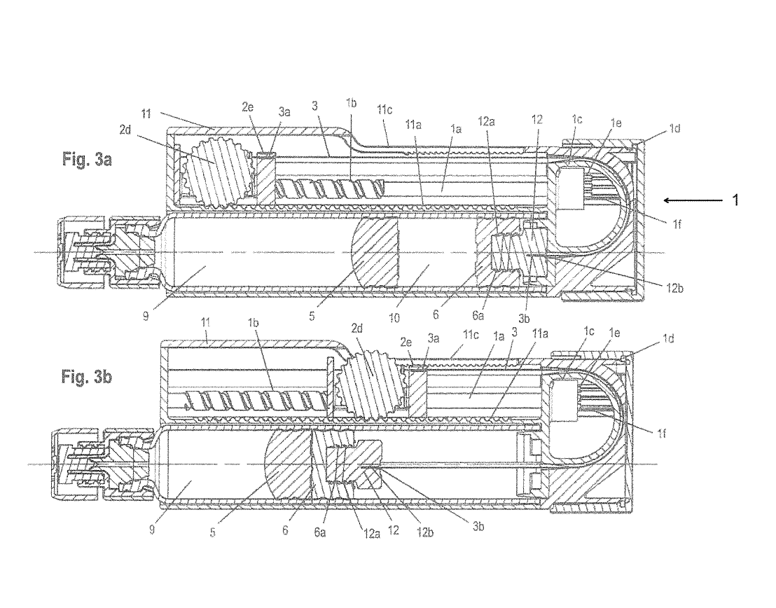

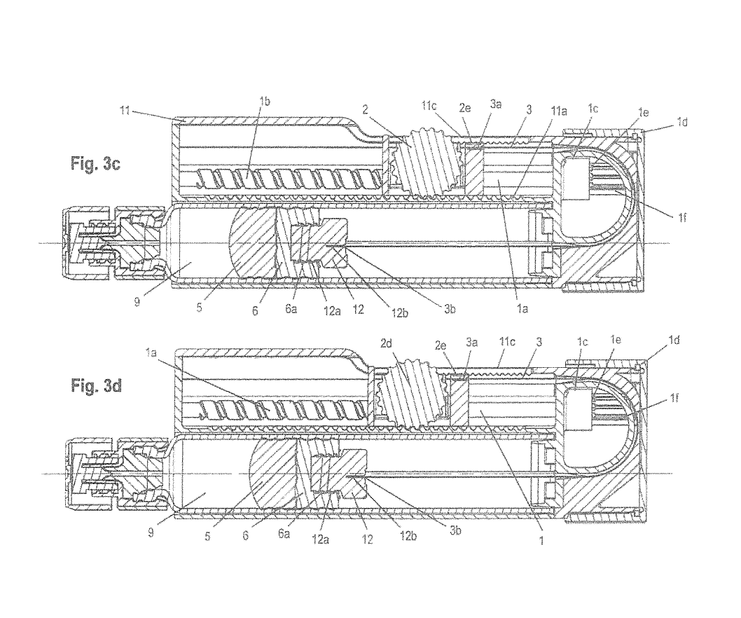

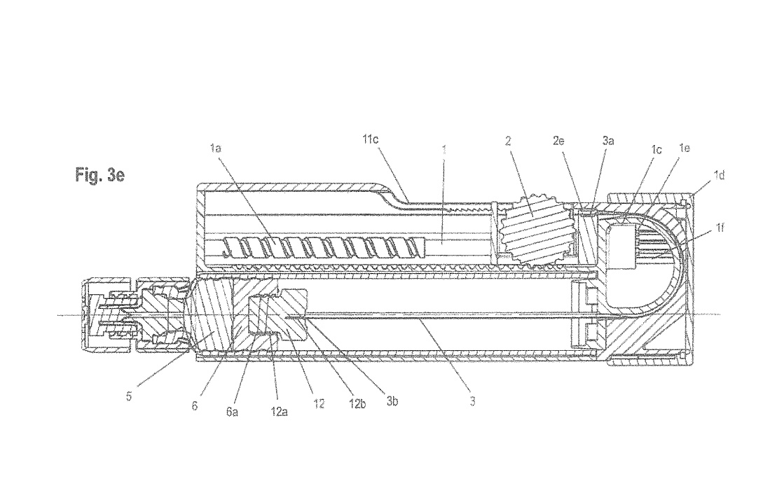

In FIG. 1, a perspective view of a first embodiment of the administration device according to the invention is represented. In FIG. 2, the first embodiment of the administration device according to the invention is represented in an exploded view. The administration device comprises a multi-chamber carpule (4) having a bypass (7) in a wall (8) of the multi-chamber carpule (4). The multi-chamber carpule (4) comprises at least one first chamber (FIG. 3a, 9) with a solid active substance and a second chamber (FIG. 3a, 10) with a solvent for the active substance, wherein the second chamber (FIG. 3a, 10) is delimited by a first plug (FIG. 3a, 5) arranged distally in the multi-chamber carpule (4) and a second plug (FIG. 3a, 6) arranged proximally in the multi-chamber carpule (4). Through the bypass (7), the solvent can be redirected from the second chamber (FIG. 3a, 10) into the first chamber (FIG. 3a, 9). The multi-chamber carpule (4) is accommodated at least partially by a housing (11) of the administration device. The housing (11) is arranged in an axially and rotationally fixed manner in relation to the multi-chamber carpule (4). The administration device moreover comprises a mixing device for mixing the active substance and the solvent. The mixing device comprises a mixing element (1), a sliding element (2), and a bendable piston rod (3). The mixing element (1) comprises a drive element (1a) with an outer thread (1b) and with an outer toothing (1c). The outer thread (1b) of the drive element (1a) extends from a distal end of the drive element (1a) in the proximal direction, wherein the outer thread (1b) is provided on only a portion of the drive element (1a). The outer thread (1b) of the drive element (1a) extends up to an aspiration stop position. The outer toothing (1c) of the drive element (1a) is in engagement with an inner toothing (1e) of a holding device (1d) of the mixing element (1). The holding device (1d) can comprise longitudinal and/or transverse webs for improved gripability by the patient. The mixing element (1) is rotatably mounted about a rotational axis in relation to the housing (11), and, for mixing the active substance and the solvent, it is rotatable about a rotational axis in a rotational direction, wherein, in particular, the rotational axis is parallel to the longitudinal axis of the device. For this purpose, the patient rotates the holding device (1d) of the mixing element (1) about the rotational axis in the rotational direction. The sliding element (2) comprises a gliding element (2a) and a gear (2d). The gliding element (2a) comprises an inner thread (2b), which is in threaded engagement with the outer thread (1b) of the mixing element (1). Furthermore, the gliding element (2a) comprises a longitudinal guide (2c), which is in engagement with a longitudinal guide provided in the interior of the housing (11). After the mixing operation, the sliding element (2), in particular the gear (2d), protrudes from a recess (11c) provided on the housing (11). The sliding element (2), in particular the gear (2d), is accessible to the patient after the mixing operation. After the mixing operation, the sliding element (2) can be moved axially back and forth in relation to the housing (11), wherein the sliding element (2) is coupled via the piston rod (3) to the second plug (6) in such a manner that, during the axial back and forth movement of the sliding element (2) in relation to the housing (11), the second plug (FIG. 3a, 6) can be moved back and forth in relation to the wall (8) of the multi-chamber carpule (4). For this purpose, a first end of the piston rod (3a) is connected in an axially fixed manner and preferably in a rotationally fixed manner to the gliding element (2a) via a fastening element (2e), for example, in the form of a slot. Alternatively, the first end of the piston rod (3a) can be connected directly or indirectly to the gliding element (2a). A second end of the piston rod (3b) is connected in an axially fixed manner and preferably in a rotationally fixed manner via a connection element (12) to the second plug (6). For this purpose, the connection element (12) comprises, on the one hand, an outer thread (12a), which is in threaded engagement with an inner thread (FIG. 3a, 6a) of the second plug (FIG. 3a, 6), and, on the other hand, a holding element (12b), for example, in the form of a slot, which is in engagement with the second end of the piston rod (3b). Alternatively, the second end of the piston rod (3) can be connected directly or indirectly to the second plug (FIG. 3a, 6). The bendable piston rod (3) can preferably be of resilient design. Particularly preferably, the bendable piston rod (3) is a resilient band, in particular a metal band made of a spring steel, for example. The housing (11) can comprise a guide slot, which can guide the bendable piston rod (3), in particular, a two-walled saddle guide which deflects the piston rod (3), in particular a band made of a spring material, by traction and pressure by approximately 180°. Alternatively or additionally, the holding device (1d) can comprise a guide slot for guiding the piston rod (3). The housing (11) moreover comprises a toothing (FIG. 3a, 11 a), which is in engagement with the gear (2d) of the sliding element (2). The gear (2d) can roll off on the toothing (FIG. 3a, 11a) of the housing (11), wherein its rotational axis is rotatably guided longitudinally in the sliding element (2) through the housing (11). The distal end of the administration device is formed in such a manner that an infusion set or a her device with a cannula can be attached so that, for example, a fluid connection can be established with a patient. On the administration device, a first detent device can be provided in such a manner that, after the completion of the mixing operation, an axial displacement of a distal end of the second plug (FIG. 3a, 6) in relation to the wall (8) of the multi-chamber carpule (4) is blocked by means of a distal end of the bypass (7) in the proximal direction. For this purpose, the sliding element (2) can comprise a detent arm (not visible), which can reach a detent groove (not visible) of the housing (11). The detent groove of the housing (11) can comprise a distal abutment (not visible) and a proximal abutment (not visible), wherein the detent arm of the sliding element (2) can be moved axially back and forth between the distal and the proximal abutment of the detent groove of the housing (11). The detent arm can be pretensioned radially outward in such a manner that it slides along the surface of the housing (11) and engages in the detent groove of the housing (11). The abutment contact between the detent arm of the sliding element (2) and the distal abutment of the detent groove defines an aspiration stop position, and the abutment contact between the detent arm of the sliding element (2) and the proximal abutment of the detent groove defines a dispensing position. The two abutment contacts can be formed as axial abutment contacts. The detent groove of the housing can be used as a longitudinal guide for the axial guiding of the sliding element (2) towards the housing (11).

The housing (11) of the administration device can moreover comprise a detent arm (11b), which is in engagement with an inner toothing (10 of the holding device (1d) for the detent arm (11b). This engagement can generate a clicking noise. The inner toothing (10 of the holding device (1d) for the detent arm (11b) of the housing (11) can be identical to the inner toothing (1e) of the holding device (1d) for the outer toothing (1c) of the drive element (1a).

Preferably, an engagement between the detent arm (11b) of the housing (11) and the inner toothing (1f) of the holding device (1d) for the detent arm (11b) can form a second detent and/or indicator device for the detent mechanism and/or the indicator of the individual preparation steps of the administration device. The detent mechanism can be formed as a reverse motion lock. The detent mechanism can preferably generate an acoustic signal, which can indicate the reaching of a corresponding position. The detent arm (11b) is designed in such a manner that, due to its pretensioning, it blocks the holding device (1d) from being rotated out again or from being rotated back in relation to the housing (11) by an abutment on the inner toothing (1f) of the holding device. From the start position, it is therefore only possible to rotate the holding element (1d) further in one rotational direction. Alternatively or additionally to the acoustic signal, a visual and/or tactile and/or other acoustic signal can be provided on the administration device.

In FIG. 3a, a longitudinal sectional view of the first embodiment of the administration device is represented in a start position of the mixing element (1). In the second chamber (10) of the multi-chamber carpule (4), the solvent is accommodated, and in the first chamber (9) of the multi-chamber carpule (4), the solid active substance is accommodated. The first plug (5) is arranged proximally offset in relation to the bypass (7). The second chamber (10) is delimited on the distal side by the first plug (5) and on the proximal side by the second plug (6). The sliding element (2) is arranged in a distal position in relation to the housing (11), wherein the sliding element (2) is accommodated by the housing (11). The sliding element (2) is not accessible to the patient. The gliding element (2a) is in threaded engagement via the inner thread (2b) with the outer thread (1b) of the drive element (1a) of the mixing element (1).

In FIG. 3b, a longitudinal sectional view of the first embodiment of the administration device of a mixing position is represented. On the distal end of the administration device, an infusion set or another device with a cannula can be placed. The holding device (1d) of the mixing element (1) is rotated about a rotational axis in a rotational direction in relation to the housing (11). Due to the toothing engagement between the inner toothing (1e) of the holding device (1d) and of the outer thread (1c) of the drive element (1a), the rotation movement of the holding device (1d) is transmitted via the threaded engagement between the outer thread (1b) of the drive element (1a) and the inner thread (2b) of the gliding element (2a) of the sliding element (2), and via the longitudinal guide (2c) of the gliding element (2a) and the housing (11) in an axial movement of the sliding element (2). The sliding element (2) moves over the toothing (11a) of the housing (11) in the proximal direction. The bendable piston rod (3) coupled to the sliding element (2) can be moved, with deflection in the radial direction, by the guide slot of the housing (11) and/or of the holding device (1d). The second piston (6) that is connected in an axially fixed manner to the piston rod (3) is also moved in the distal direction. The drive force is thereby transmitted first through the solvent in the second chamber (10) to the first plug (5), so that the two plugs (5, 6) are driven distally. As soon as the first plug (5) comes in contact in the area of the bypass (7), the first plug (5) remains at rest in relation to the wall (8) of the multi-chamber carpule (4). The second plug (6) is driven further, so that the solvent arrives from the first chamber (10) via the bypass (7) in the first chamber (5) and can dissolve the active substance located therein. The second plug (6) is driven until it strikes the first plug (5). Thereby, the mixing position of the administration device is reached. The sliding element (2), in particular the gear (2d) of the sliding element (2), can now protrude from the recess (11c) of the housing (11) and it is consequently accessible to the patient.

In FIG. 3c, a longitudinal representation of the first embodiment of the administration device of a priming position is represented. The gear (2d) of the sliding element is moved via the toothing (11a) of the housing (11) axially in the proximal direction in relation to the housing (11). The piston rod (3) is moved further in the distal direction. Here, the first plug (5) and the second plug (6) are moved in the distal direction. By means of the infusion set or the other device with a cannula, excessive air can thus escape from the first chamber (9).

Alternatively, the gear (2d) of the sliding element (2) can protrude from the recess (11c) of the housing (11) only once it is in the priming position. To reach this priming position, the holding device (1d) of the mixing element (1) therefore has to be moved further in the rotational direction.

FIG. 3d is a longitudinal sectional view of the first embodiment of the administration device in an aspiration stop position. The gliding element (2a) of the sliding element (2) is disengaged from threaded engagement with the drive element (1a) of the mixing element (1). The administration device comprises a first detent device, which is designed so that, after the completion of the mixing operation, an axial displacement of a distal end of the second plug (6) in relation to the wall (8) of the multi-chamber carpule (4) over a distal end of the bypass (7) in the proximal direction is blocked. For this purpose, a detent arm (not visible) can be provided on the sliding element (2) and reach a detent groove (not visible) of the housing (11) with a distal abutment (not visible) and a proximal abutment (not visible) of the housing (11), in order to limit a relative axial movement between the sliding element (2) and the housing (11). The abutment contact between the distal abutment of the detent groove of the housing (11) and the detent arm can define the aspiration stop position, and the abutment contact between the proximal abutment of the detent groove of the housing (11) and the detent arm can define the dispensing position. The engagement between the detent arm of the sliding element (2) and the detent groove of the housing (11) can be used for the axial guiding of the sliding element (2) towards the housing (11). The sliding element (2), in particular the gear (2d) of the sliding element (2), can be moved back and forth between the aspiration stop position and preferably before reaching the dispensing position in relation to the housing (11). The gear (2d) of the sliding element (2) is in engagement with the toothing (11a) of the housing (11). The relative back and forth movement between the sliding element (2), in particular the relative back and forth movement of the gear (2d) of the sliding element and the housing (11), should be designed in such a manner that the patient can control whether the infusion set or the other device with a cannula for the administration of the dissolved active substance has been stuck into the vein. After this control or after several such controls, or after this relative back and forth movement or several such relative back and forth movements, the patient moves the sliding element (2), preferably the gear (2d) of the sliding element (2), further in the proximal direction until the dispensing position, as can be seen in FIG. 3e, has been reached. The movement of the sliding element (2) is transmitted via the piston rod (3) and the guide slot to the first plug (5) and to the second plug (6), so that the mixed active substance is dispensed from the first chamber (9) or bodily fluid is aspired.

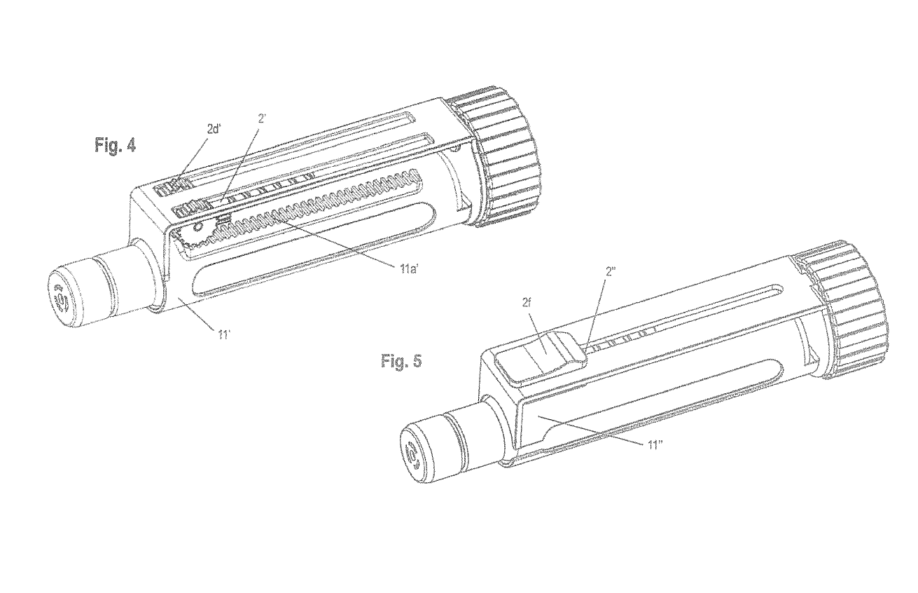

In FIG. 4, an exploded view of a second embodiment of an administration device according to the invention is represented. The administration device differs from the administration device of the first embodiment substantially only with regard to the design of the sliding element (2′) and of the housing (11′) and with regard to the interaction of the two parts during the mixing operation. The drive element (2′) comprises two gears (2d′), which collaborate with corresponding toothings (11a′) of the housing (11′). Furthermore, the housing (11′) is designed in such a manner that the two gears (2d′) are accessible to the patient during the entire mixing operation. Alternatively, the housing (11′) can be formed in such a manner that the two gears (2d′) are accessible to the patient only after the mixing operation or only after the priming operation.

In FIG. 5, a perspective view of a third embodiment of an administration device according to the invention is represented. The administration device differs from the administration device of the first embodiment substantially only with regard to the design of the sliding element (2″) and of the housing (11′) and with regard to the interaction of the two parts during the mixing operation. Instead of the gear, the sliding element (2″) comprises a sliding portion (2f), which is connected to a gliding element (not visible) of the sliding element (2″). The sliding portion (2f) is in a longitudinal guide with the housing (11″), wherein the housing (11″) comprises no toothing. Furthermore, the housing (11″) is designed in such a manner that the sliding portion (2f) is accessible to the patient during the entire mixing operation. Alternatively, the housing (11″) can be designed in such a manner that the sliding portion (2f) is accessible to the patient only after the mixing operation or only after the priming operation.

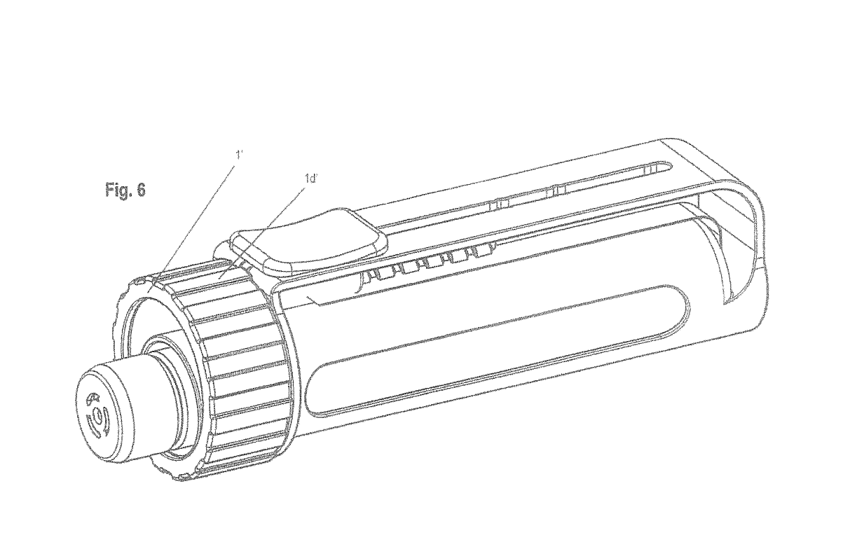

In FIG. 6, a perspective view of a fourth embodiment of an administration device according to the invention is represented. The administration device differs from the administration device of the second embodiment substantially only with regard to the arrangement of the mixing element (1′). The mixing element (1′), in particular the holding element (1d′) of the mixing element, is arranged on the distal side of the administration device.

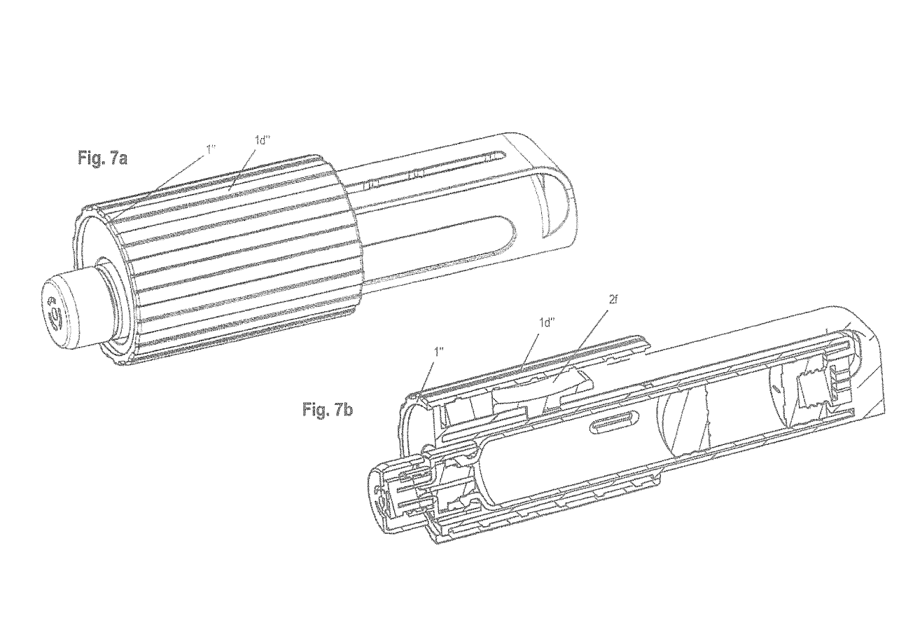

In FIG. 7a, a perspective view of a fifth embodiment of an administration device according to the invention, and, in FIG. 7b, the fifth embodiment of the administration device according to the invention are represented in a longitudinal representation. The administration device differs from the administration device of the fourth embodiment substantially only with regard to the design of the mixing element (1″), in particular of the holding device (1d″) of the mixing element (1″) and with regard to the interaction of the mixing element (1″) with the sliding element, in particular the sliding portion (2f). During the mixing operation, the holding device (1d″) covers the sliding portion (2f). After the mixing operation or alternatively after the priming operation, the sliding portion (2f) is accessible to the patient.

What is claimed is:

1. A device for administering an active substance, comprising
a multi-chamber carpule comprising a bypass in a wall of the multi-chamber carpule, at least one first chamber with a solid active substance and a second chamber with a solvent for the active substance, wherein the second chamber is delimited by a first plug arranged distally in the multi-chamber carpule and a second plug arranged proximally in the multi-chamber carpule;
a housing, wherein the multi-chamber carpule is accommodated at least partially by the housing; and
a mixing device for mixing the active substance and the solvent in a mixing operation, the mixing device comprising a mixing element, a sliding element and a bendable piston rod;
wherein the mixing element is rotatably mounted about a rotational axis in relation to the housing and for rotation about the rotational axis in a rotational direction in order to mix the active substance and the solvent in the mixing operation,
wherein, after the mixing operation, the sliding element is movable axially back and forth in relation to the housing, and
wherein the sliding element is coupled to the second plug via the piston rod in such a manner that, during the axial back and forth movement of the sliding element in relation to the housing, the second plug is movable back and forth in relation to the wall of the multi-chamber carpule,
wherein the device further comprises a detent device configured such that, after the mixing operation, an axial displacement of a distal end of the second plug in relation to the wall of the multi-chamber carpule is blocked in the proximal direction by means of a distal end of the bypass.

2. The device according to claim 1, wherein the detent device forms a first detent position between the housing and the mixing element or the sliding element or the piston rod.

3. The device according to claim 1, further comprising a second detent device, the second detent device configured such that during the mixing of the active substance and the solvent in the mixing operation, an axial displacement of the second plug in relation to the wall of the multi-chamber carpule in the proximal direction is prevented.

4. The device according to claim 3, wherein the second detent device forms a second detent position between the housing and the mixing element or the sliding element or the piston rod.

5. The device according to claim 1, wherein the bendable piston rod is configured to be resilient.

6. The device according to claim 1, wherein the sliding element is mounted axially displaceably in relation to the multi-chamber carpule.

7. The device according to claim 1, wherein the sliding element is in a longitudinal guide with the housing.

8. The device according to claim 1, wherein the mixing element further comprises a thread and the sliding element comprises a counter-thread, wherein the thread and the counter-thread are in a threaded engagement.

9. The device according to claim 1, wherein the sliding element comprises a gear configured to interact with a toothed surface arranged on a surface of the housing.

10. The device according to claim 1, wherein, during mixing of the active substance and the solvent in the mixing operation, the sliding element is accommodated by the housing.

11. A method for operating a device according to claim 1, comprising:
relative rotation of the mixing element towards the housing of the device about the rotational axis;
mixing of the solid active substance with the solvent for the active substance in the multi-chamber carpule;
axial movement of the sliding element after the mixing operation in relation to the housing; and
blocking of the axial displacement of the distal end of the second plug in relation to the wall of the multi-chamber carpule by means of the distal end of the bypass in the proximal direction after the mixing operation.

12. The method of claim 11, wherein a gear of the sliding element rolls off a toothing of the housing.

13. The method of claim 11, wherein after the completion of the mixing operation, at least a portion of the sliding element protrudes from the housing.

14. The method of claim 11, wherein, during the mixing operation, a holding element of the mixing element covers a sliding portion of the sliding element.

15. The method of claim 11, wherein the mixing element rotates about the rotational axis in only one rotational direction in relation to the housing.
